(19) United States
(12) Patent Application Publication
(10) Pub. No.: US 2023/0165529 A1
(43) Pub. Date: Jun. 1, 2023

(54) PHYSIOLOGICAL SENSING DEVICE

(71) Applicant: Industrial Technology Research Institute, Hsinchu (TW)

(72) Inventors: Yu-Hua Chung, Pingtung County (TW); Tai-Jui Wang, Kaohsiung City (TW); Chieh-Wei Feng, Taoyuan City (TW); Tzu-Yang Ting, Taipei City (TW); Jui-Wen Yang, New Taipei City (TW)

(73) Assignee: Industrial Technology Research Institute, Hsinchu (TW)

(21) Appl. No.: 17/584,394

(22) Filed: Jan. 26, 2022

(30) Foreign Application Priority Data
Nov. 30, 2021 (TW) 110144623

(51) Int. Cl. A61B 5/00; A61B 5/263; A61B 5/277; A61B 5/271

(52) U.S. Cl. CPC A61B 5/683; A61B 5/7203; A61B 5/263; A61B 5/277; A61B 5/271; A61B 2562/046

(57) ABSTRACT

A physiological sensing device is provided, including an electronic component, a coupled sensing electrode, a coupling dielectric layer, and a wire layer. The coupled sensing electrode is configured to sense a physiological signal of an object, wherein there is a capacitance value between the object and the coupled sensing electrode. The coupling dielectric layer is disposed under the coupled sensing electrode, so that the capacitance value is between 1 nF and 10 nF. The wire layer is electrically connected to the electronic component and the coupled sensing electrode.

### CROSS-REFERENCE TO RELATED APPLICATION

This application claims the priority benefit of Taiwan application serial no. 110144623, filed on Nov. 30, 2021. The entirety of the above-mentioned patent application is hereby incorporated by reference herein and made a part of this specification.

### TECHNICAL FIELD

The disclosure relates to a sensing device, and relates to a physiological sensing device.

### BACKGROUND

In terms of wearable biomedical sensing techniques, physiological signal measurement equipment (such as sensing electrode patches or sensors) may be worn on the person to be tested, and various physiological signals of the wearer may be recorded at any time in a non-invasive manner. In this way, the wearer's body temperature, pulse, heartbeat, breathing rate . . . and other physiological conditions of the human body may be known. In addition, physiological signal measuring equipment may also remind or prevent possible physiological abnormalities, and even achieve the effect of prompt and timely reminder and SOS when symptoms occur. Therefore, wearable biomedical sensing techniques are extremely convenient technological advancements for medical care (such as patients recuperating at home, patients with a history of heart disease, or elderly people living alone . . . etc.), and may also be used for real-time monitoring of physiological status during exercise. Moreover, in addition to protective gear or fabric, etc. that may be worn on the person to be tested, wearable biomedical sensing devices may also include vehicle-mounted devices (such as seats or seat belts) and long-term care devices (such as wheelchairs or mattresses).

However, due to the limitations of the prior art, a sensing electrode patch needs to be tightly attached to the wearer's skin, and prolonged wear may cause the wearer to experience tight pressing, discomfort, or allergies. Based on this, a new type of coupled physiological signal sensing device may reduce the feeling of tight pressing during wear. However, there are issues with weak coupling physiological signals, noise interference/product durability, variations in spacer material and thickness, and sweat interfering with signals. More specifically, although the coupled physiological sensing method may solve the discomfort caused by the conventional impedance sensing, coupling of physiological signals causes the gap between the sensing electrode and the skin to become larger due to different use situations (low pressure, non-contact) and causes signal drop, or the signal is distorted through the circuit, thus affecting the interpretation of the physiological signals.

Based on the above, how to improve the coupling capacitance of the coupled physiological signal sensing device and at the same time achieve module conformability, increase sensing signal strength, and alleviate the issue of peeling in manufacture caused by the mismatch of stress of the sensing structure have become increasingly important.

### SUMMARY

A physiological sensing device of an embodiment of the disclosure includes an electronic component, a coupled sensing electrode, a coupling dielectric layer, and a wire layer. The coupled sensing electrode is configured to sense a physiological signal of an object, wherein there is a capacitance value between the object and the coupled sensing electrode. The coupling dielectric layer is disposed under the coupled sensing electrode, so that the capacitance value is between 1 nF and 10 nF. The wire layer is electrically connected to the electronic component and the coupled sensing electrode.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A and FIG. 1B are schematic cross-sectional views of a physiological sensing device according to the first embodiment of the disclosure.

FIG. 2 is a schematic cross-sectional view of a physiological sensing device according to the second embodiment of the disclosure.

FIG. 3 is a schematic cross-sectional view of a physiological sensing device according to the third embodiment of the disclosure.

FIG. 4 is a schematic cross-sectional view of a physiological sensing device according to the fourth embodiment of the disclosure.

FIG. 5 is a schematic cross-sectional view of a physiological sensing device according to the fifth embodiment of the disclosure.

FIG. 6 is a schematic cross-sectional view of a physiological sensing device according to the sixth embodiment of the disclosure.

### DETAILED DESCRIPTION OF DISCLOSED EMBODIMENTS

Embodiments are provided hereinafter and described in detail with reference to figures. However, the embodiments provided are not intended to limit the scope of the disclosure. Moreover, the figures are only descriptive and are not drawn to scale. For ease of explanation, the same elements below are provided with the same reference numerals. Moreover, terms such as “comprise”, “include”, and “have” used in the specification are all open terms, i.e., “comprise, but not limited to”. Moreover, directional terms used in the specification such as “up” and “down” are directions used in the figures and are not used to limit the disclosure. Moreover, the quantities and shapes mentioned in the specification are used to specifically describe the disclosure in order to understand the content thereof, and not to limit the disclosure.

An embodiment of the disclosure provides a physiological sensing device that may effectively increase coupling capacitance and at the same time achieve module conformability, thus enhancing sensing signal strength and alleviating the issue of peeling in manufacture caused by the mismatch of stress of the sensing structure.

An embodiment of the disclosure provides a physiological sensing device including a coupling dielectric layer disposed under a coupled sensing electrode. The coupling dielectric layer may increase the capacitance value and compensate for the attenuation of the coupling signal caused by the coupling spacing between the sensing electrode and an object to be measured, so as to effectively increase sensing signal strength. The physiological sensing device of an embodiment of the disclosure may also include a stress compensation layer disposed above the coupling dielectric layer. The stress compensation layer may alleviate the issues of high stress and the brittle characteristic of the material itself of the coupling dielectric layer or the stress matching of the sensing structure. In this way, the physiological sensing device of an embodiment of the disclosure may effectively increase the coupling capacitance and at the same time achieve module conformability, and also increase sensing signal strength.

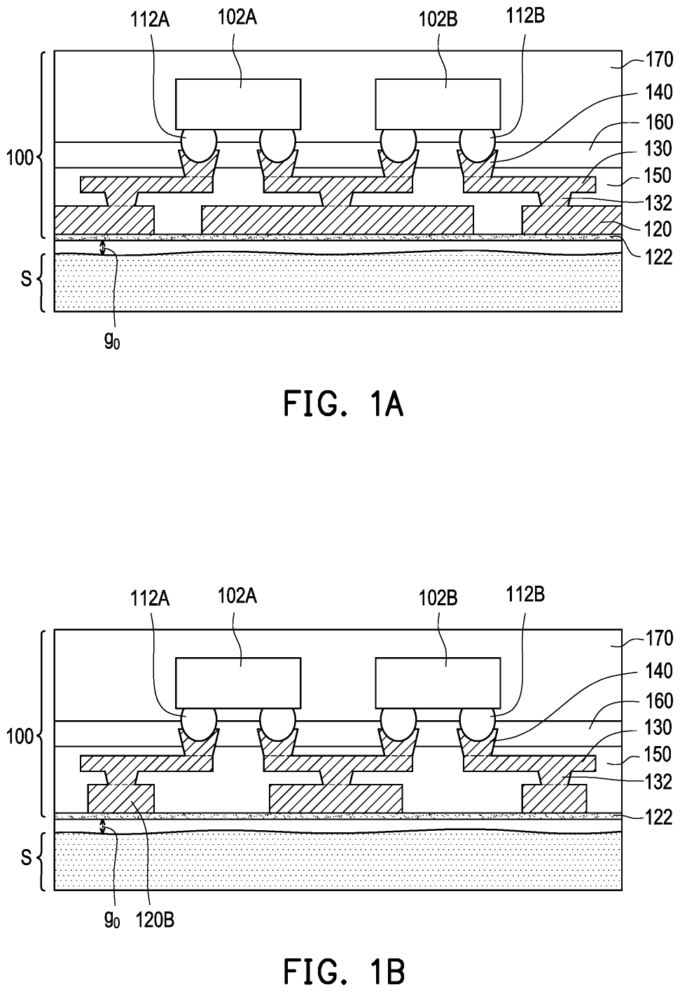

FIG. 1A and FIG. 1B are schematic cross-sectional views of a physiological sensing device according to the first embodiment of the disclosure.

Referring to FIG. 1A, a physiological sensing device 100 may include a first electronic component 102A, a second electronic component 102B, conductive terminals 112A and 112B, a coupled sensing electrode 120, a coupling dielectric layer 122, a noise isolation layer 130, a conductive via 132, a wire layer 140, a dielectric layer 150, a coupling stress adjustment layer 160, and a packaging structure 170. The first electronic component 102A may include, for example, a high-impedance front-end circuit module, and the second electronic component 102B may include, for example, an integrated circuit (IC). Each of the first electronic component 102A and the second electronic component 102B has at least one conductive terminal 112A and 112B. The conductive terminals 112A and 112B may include solder balls, for example, and the first electronic component 102A and the second electronic component 102B may be electrically connected to other components via the conductive terminals 112A and 112B. The coupled sensing electrode 120 is configured to sense a physiological signal of an object, wherein there is a capacitance value between the object and the coupled sensing electrode 120. The object may include, for example, a skin S. The coupling dielectric layer 122 is disposed under the coupled sensing electrode 120. The noise isolation layer 130 is disposed above the coupled sensing electrode 120. The coupled sensing electrode 120 and the noise isolation layer 130 are isolated by the dielectric layer 150 and the projections thereof are overlapped with each other. The noise isolation layer 130 is electrically connected to the coupled sensing electrode 120 via the conductive via 132. In the present embodiment, the noise isolation layer 130 may include a ground circuit or a ground patch, and the noise isolation layer 130 may prevent the coupled sensing electrode 120 from directly receiving an external noise source and resulting in a physiological signal-to-noise ratio (SNR) that is too small for precise sensing. The wire layer 140 is disposed above the noise isolation layer 130. The wire layer 140 and the noise isolation layer 130 are isolated by the dielectric layer 150 and are electrically connected to the first electronic component 102A, the second electronic component 102B, and the coupled sensing electrode 120, respectively. The wire layer 140 is, for example, electrically connected to the first electronic component 102A and the second electronic component 102B via the conductive terminals 112A and 112B, respectively. The layout design may be adjusted according to design requirements, and is not limited in the disclosure. The coupling stress adjustment layer 160 is disposed above the dielectric layer 150, and the coupling stress adjustment layer 160 may improve the overall stress matching of the physiological sensing device 100. The packaging structure 170 is disposed above the first electronic component 102A and the second electronic component 102B and covers the first electronic component 102A and the second electronic component 102B. The material of the packaging structure 170 is, for example, a soft material, and may include polydimethylsiloxane (PDMS) or thermoplastic polyurethane (TPU), and is configured to provide protection and insulation effects to the first electronic component 102A and the second electronic component 102B.

In the present embodiment, the material of the coupled sensing electrode 120 may include Mo, Ti, Al, or Cu, for example, and the thickness is, for example, 300 nm to 5000 nm.

In the present embodiment, the material of the coupling dielectric layer 122 may include, for example, SiO₂, Si₃N₄, Al₂O₃, TiO₂, or polyimide. The thickness of the coupling dielectric layer 122 is, for example, 0.1 μm to 10 μm. By adjusting the thickness of the coupling dielectric layer 122 and using a dielectric constant material, the capacitance value may be increased so that the capacitance value is between 1 nF and 10 nF. In one embodiment, the dielectric constant of the dielectric constant material is, for example, 3 to 110. In another embodiment, the dielectric constant of the dielectric constant material is, for example, 7 to 90.

In more detail, the capacitance value C may be calculated and adjusted via the following equation:

$$C = \frac{{ε}_{0} A}{{h}_{1} \frac{{ε}_{0}}{{ε}_{d}} + {g}_{0}}$$

In the equation, the definition of each symbol is as follows:
ε₀: 8.85×10⁻¹²
A: area of coupled sensing electrode
h₁: thickness of coupling dielectric layer
ε_{d}: dielectric constant of coupling dielectric layer
Coupling spacing g₀: coupling spacing between coupling dielectric layer and skin
(when tightly attached to the skin, the coupling spacing g₀ may be 0), when the coupling capacitance value is less than 1.1 nF, the (signal/noise) coupling rate is less than 50%. If the coupled sensing electrode uses PI as the coupling dielectric layer, the area A of the coupled sensing electrode should be increased to 6.283 cm² (r=2 cm circular electrode, r is the diameter).

In the following Table 1, Example 1, Example 2, and Comparative Example 1 are used to illustrate the difference in capacitance values between 1 nF and 10 nF or outside this range. Generally speaking, the higher the signal-to-noise ratio, the higher the signal quality.

TABLE 1

| | | Thickness | Dielectric | | | | |
|---|---|---|---|---|---|---|---|
| | Area A of | h1 of | constant ε_{d} | | | | |
| | coupled | coupling | of coupling | Coupling | | | |
| | sensing | dielectric | dielectric | spacing | Capacitance | Coupling | Signal-to- |
| | electrode | layer | layer | (g₀) | C | rate | noise ratio |
| Example 1 | 3.14 cm² | 205 nm | 7(SiNx) | 1.2 μm to | 1.1 nF to | 53.8% to | >20 dB |
| | | | | 2.5 μm | 2.2 nF | 63% | |
| Example 2 | 6.28 cm² | 15 μm | 3.2 (PI) | 1.2 μm to | 0.79 pF to | 45% to | 10 dB to |
| | | | | 2.5 μm | 0.97 pF | 50% | 19 dB |
| Comparative | 3.14 cm² | 10 μm | 3.2(PI) | 1.2 μm to | 0.50 nF to | 20% to | <10 dB |
| Example | | | | 2.5 μm | 0.66 nF | 40% | |

Please refer to FIG. 1B. In the physiological sensing device of FIG. 1B, the elements and the technical content thereof are substantially similar to those of the physiological sensing device of FIG. 1A. Therefore, the same reference numerals are used to denote the same or similar elements, and the description of the same technical content is omitted. The difference between FIG. 1B and FIG. 1A is that the projection area of the noise isolation layer 130 covers the projection area of the coupled sensing electrode 120B. In other words, the projection area of the noise isolation layer 130 may be greater than or equal to the projection area of the coupled sensing electrode 120B.

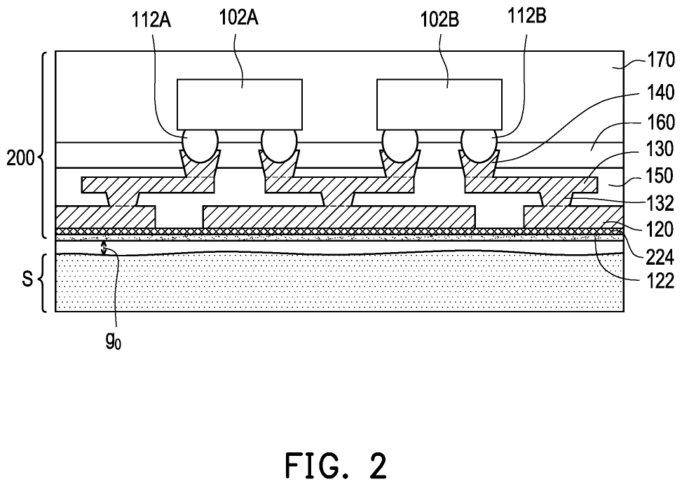

FIG. 2 is a schematic cross-sectional view of a physiological sensing device according to the second embodiment of the disclosure. The embodiment shown in FIG. 2 is similar to the first embodiment shown in FIG. 1A. Therefore, the following embodiment adopts the reference numerals and part of the content of the previous embodiment, wherein the same reference numerals are used to represent the same or similar elements and descriptions of the same technical content are omitted. The omitted portions are as described in the embodiments above and are not repeated in the embodiments below.

Referring to FIG. 2, in the present embodiment, the difference from the first embodiment is that a physiological sensing device 200 may further include a stress compensation layer 224 disposed between the coupled sensing electrode 120 and the coupling dielectric layer 122 to improve the stress of the coupling dielectric layer 122. However, the disclosure is not limited thereto. The stress compensation layer 224 may also be disposed under the coupling dielectric layer 122 to improve the stress of the coupling dielectric layer 122. The material of the stress compensation layer 224 may be an inorganic material, and may include, for example, Pb(ZrTi)O₃, SiO₂, ZnO, Ta₂O₅, Si₃N₄, SiON, BaTiO₃, CaTiO₃, SrTiO₃, TiO₂, MgO, AlN, or Al₂O₃. The stress compensation layer 224 may alleviate the high stress and brittle characteristic of the material itself of the coupling dielectric layer 122 or the stress matching with other film layers, such as the coupled sensing electrode 120, the noise isolation layer 130, the wire layer 140, etc., so as to prevent peeling issues in manufacture. In the present embodiment, the stress value of the stress compensation layer 224 may be adjusted in consideration of the stress value of the physiological sensing device 200. For example, when the stress value of the physiological sensing device 200 is, for example, 50 MPa to 200 MPa (tensile stress), the physiological sensing device 200 is curled, and the stress value of the stress compensation layer 224 may be adjusted to, for example, −50 MPa to −200 MPa, so as to make the physiological sensing device 200 flat; when the stress value of the physiological sensing device 200 is, for example, −50 MPa to −200 MPa (compressive stress), the physiological sensing device 200 is curled, and the stress value of the stress compensation layer 224 may be adjusted to, for example, 50 MPa to 200 MPa, so as to make the physiological sensing device 200 flat. At the same time, the coupling capacitance may still maintain 1 nF to 10 nF. In more detail, the capacitance value C may be calculated and adjusted via the following equation:

$$C = \frac{{ε}_{0} A}{{h}_{1} \frac{{ε}_{0}}{{ε}_{d}} + {g}_{0}}$$

In the equation, the definition of each symbol is as follows:
ε₀: 8.85×10⁻¹²
A: area of coupled sensing electrode
h₁: thickness of coupling dielectric layer
ε_{d}: dielectric constant of coupling dielectric layer
Coupling spacing g₀: coupling spacing between coupling dielectric layer and skin
(when tightly attached to the skin, the coupling spacing g₀ may be 0)

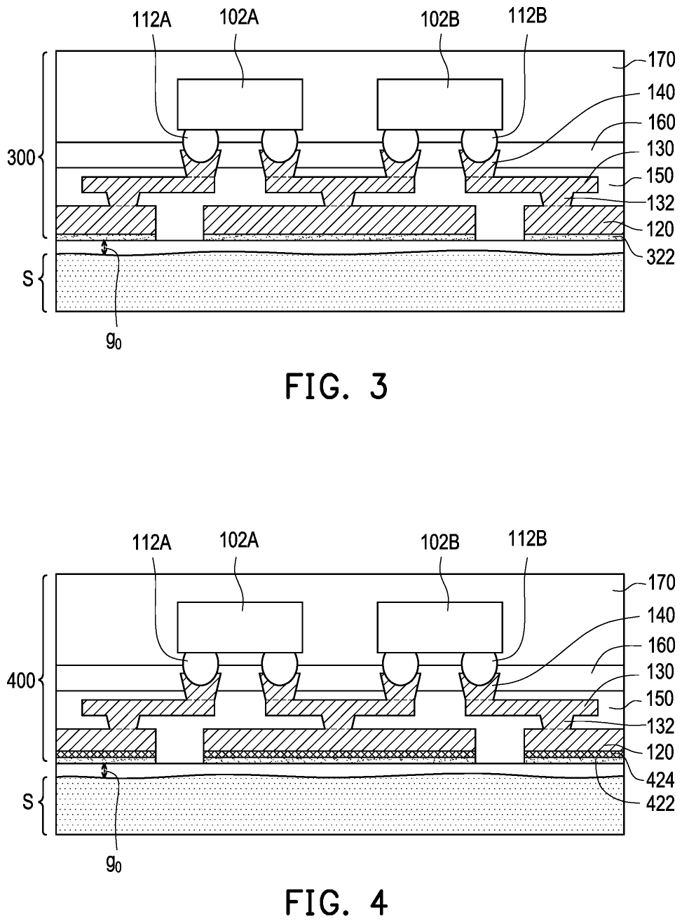

FIG. 3 is a schematic cross-sectional view of a physiological sensing device according to the third embodiment of the disclosure. The embodiment shown in FIG. 3 is similar to the first embodiment shown in FIG. 1A. Therefore, the following embodiment adopts the reference numerals and part of the content of the previous embodiment, wherein the same reference numerals are used to represent the same or similar elements and descriptions of the same technical content are omitted. The omitted portions are as described in the embodiments above and are not repeated in the embodiments below.

Referring to FIG. 3, in the present embodiment, the difference from the first embodiment is that a coupling dielectric layer 322 of a physiological sensing device 300 adopts a patterned design to reduce the excessive internal stress of the coupling dielectric layer 322, so as to avoid the issue of peeling in manufacture, and the coupling capacitance may still maintain 1 nF to 10 nF. Properties such as material of the coupling dielectric layer 322 are similar to those of the first embodiment above, and are therefore not repeated herein. It should be mentioned that, although in FIG. 3, the edges of the coupling dielectric layer 322 and the coupled sensing electrode 120 are aligned with each other, preferably, for example, the length of the coupling dielectric layer 322 is slightly protruded beyond the edge of the coupled sensing electrode 120. That is, the projection area of the coupling dielectric layer 322 of a patterned design may be greater than or equal to the projection area of the coupled sensing electrode 120. In more detail, the capacitance value C may be calculated and adjusted via the following equation:

$$C = \frac{{ε}_{0} A}{{h}_{1} \frac{{ε}_{0}}{{ε}_{d}} + {g}_{0}}$$

In the equation, the definition of each symbol is as follows:
ε₀: 8.85×10⁻¹²
A: area of coupled sensing electrode
h₁: thickness of coupling dielectric layer
ε_{d}: dielectric constant of coupling dielectric layer
Coupling spacing g₀: coupling spacing between coupling dielectric layer and skin
(when tightly attached to the skin, the coupling spacing g₀ may be 0)

FIG. 4 is a schematic cross-sectional view of a physiological sensing device according to the fourth embodiment of the disclosure. The embodiment shown in FIG. 4 is similar to the first embodiment shown in FIG. 1A. Therefore, the following embodiment adopts the reference numerals and part of the content of the previous embodiment, wherein the same reference numerals are used to represent the same or similar elements and descriptions of the same technical content are omitted. The omitted portions are as described in the embodiments above and are not repeated in the embodiments below.

Referring to FIG. 4, in the present embodiment, the difference from the first embodiment is that a physiological sensing device 400 may further include a stress compensation layer 424 disposed between the coupled sensing electrode 120 and the coupling dielectric layer 422 to improve the stress of the coupling dielectric layer 422, and both the stress compensation layer 424 and the coupling dielectric layer 422 adopt a patterned design. However, the disclosure is not limited thereto. The stress compensation layer 424 may also be disposed under the coupling dielectric layer 422 to improve the stress of the coupling dielectric layer 422. Properties such as material of the coupling dielectric layer 422 are similar to those of the first embodiment above, and properties such as material of the stress compensation layer 424 are similar to those of the second embodiment above, and are therefore not repeated herein. At this time, the coupling capacitance may still maintain 1 nF to 10 nF. In more detail, the capacitance value C may be calculated and adjusted via the following equation:

$$C = \frac{{ε}_{0} A}{{h}_{1} \frac{{ε}_{0}}{{ε}_{d}} + {g}_{0}}$$

In the equation, the definition of each symbol is as follows:
ε₀: 8.85×10⁻¹²
A: area of coupled sensing electrode
h₁: thickness of coupling dielectric layer
ε_{d}: dielectric constant of coupling dielectric layer
Coupling spacing g₀: coupling spacing between coupling dielectric layer and skin
(when tightly attached to the skin, the coupling spacing g₀ may be 0)

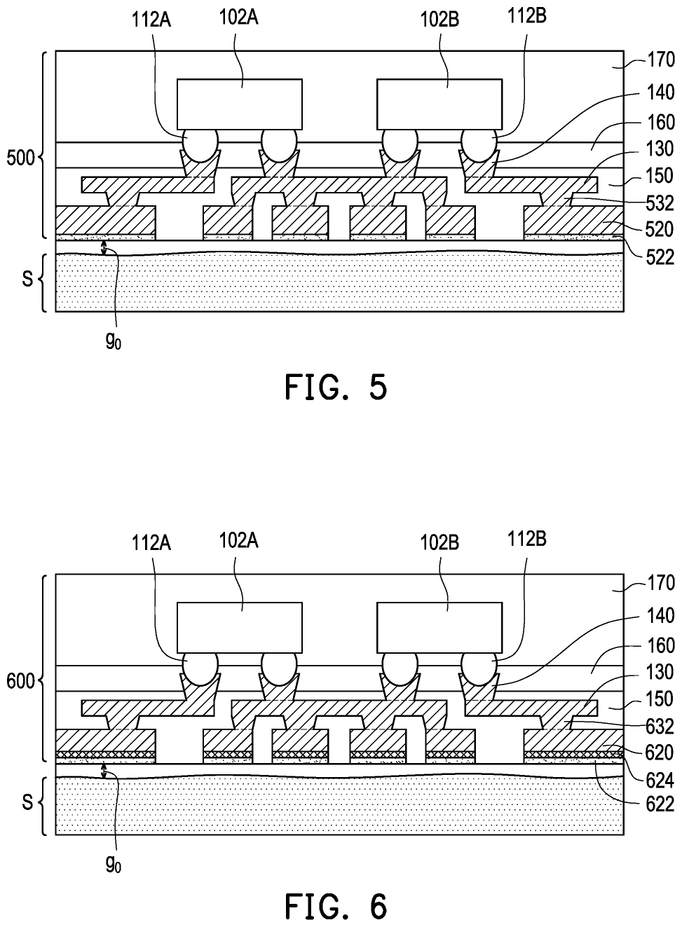

FIG. 5 is a schematic cross-sectional view of a physiological sensing device according to the fifth embodiment of the disclosure. The embodiment shown in FIG. 5 is similar to the first embodiment shown in FIG. 1A. Therefore, the following embodiment adopts the reference numerals and part of the content of the previous embodiment, wherein the same reference numerals are used to represent the same or similar elements and descriptions of the same technical content are omitted. The omitted portions are as described in the embodiments above and are not repeated in the embodiments below.

Referring to FIG. 5, in the present embodiment, the difference from the first embodiment is that a coupled sensing electrode 520 of a physiological sensing device 500 adopts an array design, and the noise isolation layer 130 is electrically connected to the coupled sensing electrode 520 via a conductive via 532. In addition, the coupling dielectric layer 522 adopts a patterned design to reduce excessive internal stress of the coupling dielectric layer 522 and avoid peeling issues in manufacture, and the coupling capacitance may still maintain 1 nF to 10 nF. Properties such as material of the coupling dielectric layer 522 are similar to those of the first embodiment above, and are therefore not repeated herein. It should be mentioned that, although in FIG. 5, the edges of the coupling dielectric layer 522 and the coupled sensing electrode 520 are aligned with each other, preferably, for example, the length of the coupling dielectric layer 522 is slightly protruded beyond the edge of the coupled sensing electrode 520. In more detail, the capacitance value C may be calculated and adjusted via the following equation:

$$C = \frac{{ε}_{0} A}{{h}_{1} \frac{{ε}_{0}}{{ε}_{d}} + {g}_{0}}$$

In the equation, the definition of each symbol is as follows:
ε₀: 8.85×10⁻¹²
A: area of coupled sensing electrode
h₁: thickness of coupling dielectric layer
ϵ_{d}: dielectric constant of coupling dielectric layer
Coupling spacing g₀: coupling spacing between coupling dielectric layer and skin
(when tightly attached to the skin, the coupling spacing g₀ may be 0)

FIG. 6 is a schematic cross-sectional view of a physiological sensing device according to the sixth embodiment of the disclosure. The embodiment shown in FIG. 6 is similar to the first embodiment shown in FIG. 1A. Therefore, the following embodiment adopts the reference numerals and part of the content of the previous embodiment, wherein the same reference numerals are used to represent the same or similar elements and descriptions of the same technical content are omitted. The omitted portions are as described in the embodiments above and are not repeated in the embodiments below.

Referring to FIG. 6, in the present embodiment, the difference from the first embodiment is that a coupled sensing electrode 620 of a physiological sensing device 600 adopts an array design, and the noise isolation layer 130 is electrically connected to the coupled sensing electrode 620 via a conductive via 632. Moreover, the physiological sensing device 600 may further include a stress compensation layer 624 disposed between the coupled sensing electrode 620 and the coupling dielectric layer 622 to improve the stress of the coupling dielectric layer 622. Moreover, both the stress compensation layer 624 and the coupling dielectric layer 622 adopt a patterned design, and the arrangement of the stress compensation layer 624 and the coupling dielectric layer 622 may correspond to the array design of the coupled sensing electrode 620. However, the disclosure is not limited thereto. The stress compensation layer 624 may also be disposed under the coupling dielectric layer 622 to improve the stress of the coupling dielectric layer 622. Properties such as material of the coupling dielectric layer 622 are similar to those of the first embodiment above, and properties such as material of the stress compensation layer 624 are similar to those of the second embodiment above, and are therefore not repeated herein. At this time, the coupling capacitance may still maintain 1 nF to 10 nF. In more detail, the capacitance value C may be calculated and adjusted via the following equation:

$$C = \frac{{ε}_{0} A}{{h}_{1} \frac{{ε}_{0}}{{ε}_{d}} + {g}_{0}}$$

In the equation, the definition of each symbol is as follows:
ε₀: 8.85×10⁻¹²
A: area of coupled sensing electrode
h₁: thickness of coupling dielectric layer
ε_{d}: dielectric constant of coupling dielectric layer
Coupling spacing g₀: coupling spacing between coupling dielectric layer and skin
(when tightly attached to the skin, the coupling spacing g₀ may be 0)

Based on the above, an embodiment of the disclosure provides a physiological sensing device including a coupling dielectric layer disposed under the coupled sensing electrode. By adjusting the thickness of the coupling dielectric layer and further using a dielectric constant material (the dielectric constant is, for example, 3 to 110, or in another embodiment, for example, 7 to 90), the capacitance value may be increased (increased to 1 nF to 10 nF) to compensate for the attenuation of the coupling signal caused by the coupling spacing between the sensing electrode and the object to be measured, so as to effectively increase the sensing signal strength. The physiological sensing device of the disclosure may also include a stress compensation layer disposed above or under the coupling dielectric layer. The stress compensation layer may alleviate the high stress and the brittle characteristic of the material itself of the coupling dielectric layer or the stress matching of the sensing structure to avoid peeling issues in manufacture, and the coupling capacitance may still maintain 1 nF to 10 nF. In addition, the physiological sensing device of the disclosure may also include a coupling dielectric layer of a patterned design to reduce excessive internal stress of the coupling dielectric layer and avoid peeling issues in manufacture, and the coupling capacitance may still maintain 1 nF to 10 nF.

It will be apparent to those skilled in the art that various modifications and variations may be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A physiological sensing device, comprising:
an electronic component;
a coupled sensing electrode configured to sense a physiological signal of an object, wherein there is a capacitance value between the object and the coupled sensing electrode;
a coupling dielectric layer disposed under the coupled sensing electrode, so that the capacitance value is between 1 nF and 10 nF; and
a wire layer electrically connected to the electronic component and the coupled sensing electrode.

2. The physiological sensing device of claim 1, wherein the object comprises a skin.

3. The physiological sensing device of claim 1, further comprising a noise isolation layer disposed above the coupled sensing electrode, and the coupled sensing electrode and the noise isolation layer are isolated by a dielectric layer and projections thereof are overlapped with each other.

4. The physiological sensing device of claim 3, wherein the wire layer is disposed above the noise isolation layer, and the wire layer and the noise isolation layer are isolated by the dielectric layer and electrically connected to the electronic component and the coupled sensing electrode, respectively.

5. The physiological sensing device of claim 1, further comprising a noise isolation layer disposed above the coupled sensing electrode, wherein the coupled sensing electrode and the noise isolation layer are isolated by a dielectric layer, and a projection area of the noise isolation layer is greater than or equal to a projection area of the coupled sensing electrode.

6. The physiological sensing device of claim 1, wherein a material of the coupling dielectric layer comprises SiO₂, Si₃N₄, Al₂O₃, TiO₂, or polyimide.

7. The physiological sensing device of claim 1, further comprising a stress compensation layer disposed between the coupled sensing electrode and the coupling dielectric layer.

8. The physiological sensing device of claim 1, wherein a material of the stress compensation layer comprises Pb(ZrTi)O₃, SiO₂, ZnO, Ta₂O₅, Si₃N₄, SiON, BaTiO₃, CaTiO₃, SrTiO₃, TiO₂, MgO, AlN, or Al₂O₃.

9. The physiological sensing device of claim 7, wherein when a stress value of the physiological sensing device is 50 MPa to 200 MPa (tensile stress), a stress value of the stress compensation layer is adjusted to −50 MPa to −200 MPa.

10. The physiological sensing device of claim 7, wherein when a stress value of the physiological sensing device is −50 MPa to −200 MPa (compressive stress), a stress value of the stress compensation layer is adjusted to 50 MPa to 200 MPa.

11. The physiological sensing device of claim 1, wherein a thickness of the coupling dielectric layer is 0.1 μm to 10 μm.

12. The physiological sensing device of claim 1, wherein the coupling dielectric layer adopts a patterned design.

13. The physiological sensing device of claim 12, further comprising a stress compensation layer disposed between the coupled sensing electrode and the coupling dielectric layer, and the stress compensation layer adopts a patterned design.

14. The physiological sensing device of claim 13, wherein a material of the stress compensation layer comprises Pb(ZrTi)O₃, SiO₂, ZnO, Ta₂O₅, Si₃N₄, SiON, BaTiO₃, CaTiO₃, SrTiO₃, TiO₂, MgO, AlN, or Al₂O₃.

15. The physiological sensing device of claim 12, wherein the coupled sensing electrode adopts an array design.

16. The physiological sensing device of claim 15, further comprising a stress compensation layer disposed between the coupled sensing electrode and the coupling dielectric layer, and the stress compensation layer adopts a patterned design.

17. The physiological sensing device of claim 16, wherein a material of the stress compensation layer comprises Pb(ZrTi)O₃, SiO₂, ZnO, Ta₂O₅, Si₃N₄, SiON, BaTiO₃, CaTiO₃, SrTiO₃, TiO₂, MgO, AlN, or Al₂O₃.

18. The physiological sensing device of claim 1, wherein the electronic component has at least one conductive terminal, and the wire layer is electrically connected to the electronic component via the at least one conductive terminal.

19. The physiological sensing device of claim 1, wherein a material of the coupled sensing electrode comprises Mo, Ti, Al, or Cu.
